(19) United States
(12) Patent Application Publication
(10) Pub. No.: US 2011/0109879 A1
(43) Pub. Date: May 12, 2011

(54) MULTIVARIATE DYNAMIC PROFILING SYSTEM AND METHODS

(76) Inventors: Daphna Palti-Wasserman, Haifa (IL); Yoram Wasserman, Haifa (IL); Noa Urman, Carcur (IL)

(21) Appl. No.: 12/942,129

(22) Filed: Nov. 9, 2010

Related U.S. Application Data
(60) Provisional application No. 61/259,512, filed on Nov. 9, 2009.

(51) Int. Cl. A61B 3/113; A61B 3/00; A61B 3/10

(52) U.S. Cl. 351/209; 351/246; 351/205

(57) ABSTRACT

There provided herein a system for profiling a personal aspect of a subject, the system comprising a processor adapted to select at least one visual stimulus from a database comprising a multiplicity of visual stimuli and at least one evoking stimulus from a database comprising a multiplicity of evoking stimuli; and at least one sensor adapted to acquire at least one eye response of a subject to said visual stimulus; wherein said processor is further adapted to perform processing and analysis of said visual stimulus, said evoking stimulus and said eye response, for profiling at least one personal aspect of said subject.

### FIELD OF DISCLOSURE

The present disclosure relates in general to the field of identification. More specifically, it relates to a system and method for identifying a subject's personal aspects.

### BACKGROUND

A variety of markets and applications require a method and system to identify a subjects identity and/or some of his personal aspects and state of mind. There is still a need in the art for more efficient and reliable identification system and methods that would allow identification of a subject and/or determining his or her personal aspects, such as state of mind, level of stress, anxiety, etc.

### SUMMARY

The following embodiments and aspects thereof are described and illustrated in conjunction with systems, tools and methods which are meant to be exemplary and illustrative, not limiting in scope.

There is provided, in accordance with some embodiments, a method for profiling a personal aspect of a subject, the method comprising the steps of subjecting a subject to at least one visual stimulus selected from a stimulus database comprising a multiplicity of stimuli and to at least one evoking stimulus selected from a database of evoking stimuli, acquiring at least one eye response from the subject to the visual stimulus and processing the eye response, the visual stimulus and the evoking stimulus for profiling at least one personal aspect of the subject. The evoking stimulus and the visual stimulus may be the same stimulus. According to some embodiments, features may be extracted from the eye response. According to some embodiments, processing may include using a class database. The class database may include either a generic baseline, or an intrinsic baseline or a personal enrolled baseline or any combination thereof. According to some embodiments, the processing of the eye response and the visual stimulus comprises pattern recognition analysis.

According to some embodiments, the visual stimulus may include a target moving and halting in a predefined trajectory.

According to some embodiments, the eye response may include fixation, gaze, saccades, convergence, rolling, pursuit, nystagmus, drift and microsaccades, physiological nystagmus pupil size, pupil dynamic, blinking or any combination thereof.

According to some embodiments, the personal aspect may include state of mind, level of stress, intensions, anxiety, fear, attentiveness, dislike, alertness, honesty, talents, concentration level, personal characteristics, emotions, preferences, mentality, drunkenness level, toxic level, background/memories or any combination thereof.

According to some embodiments, the processing of the eye response and the visual stimulus may be used for identifying a user and profiling at least one personal aspect of the subject.

There is provided, in accordance with some embodiments, a system for profiling a personal aspect of a subject, the system comprising a processor adapted to select at least one visual stimulus from a database comprising a multiplicity of visual stimuli and at least one evoking stimulus from a database comprising a multiplicity of evoking stimuli and at least one sensor adapted to acquire at least one eye response of a subject to the visual stimulus, wherein the processor is further adapted to perform processing and analysis of the visual stimulus, the evoking stimulus and the eye response, for profiling at least one personal aspect of the subject. The evoking stimulus and the visual stimulus may be the same stimulus.

According to some embodiments, the processor may further be adapted to extract features from the eye response. According to some embodiments, the processor may further be adapted to use a class database. The class database may include either a generic baseline, or an intrinsic baseline or a personal enrolled baseline or any combination thereof. The processor may further be adapted to perform pattern recognition analysis.

According to some embodiments, the visual stimulus may include a target moving and halting in a predefined trajectory.

According to some embodiments, the eye response may include fixation, gaze, saccades, convergence, rolling, pursuit, nystagmus, drift and microsaccades, physiological nystagmus pupil size, pupil dynamic, blinking or any combination thereof.

According to some embodiments, the personal aspect may include state of mind, level of stress, intensions, anxiety, fear, attentiveness, dislike, alertness, honesty, talents, concentration level, personal characteristics, emotions, preferences, mentality, drunkenness level, toxic level, background/memories or any combination thereof.

According to some embodiments, the processor may further be adapted to establish the subject's identity and to profile at least one personal aspect of the subject

### BRIEF DESCRIPTION OF THE FIGURES

Exemplary embodiments are illustrated in referenced figures. It is intended that the embodiments and figures disclosed herein are to be considered illustrative, rather than restrictive. The disclosure, however, both as to organization and method of operation, together with objects, features, and advantages thereof, may best be understood by reference to the following detailed description when read with the accompanying figures, in which:

FIG. 1 schematically illustrates a general block diagram of the system and method for profiling personal aspects of a subject, according to some embodiments of the disclosure.

FIG. 2 shows an example of changes in an eye movement response pattern in response to stress.

### DETAILED DESCRIPTION OF THE INVENTION

The following embodiments and aspects thereof are described and illustrated in conjunction with systems, tools and methods which are meant to be exemplary and illustrative, not limiting in scope.

There are provided herein, in accordance with some embodiments an innovative system and method to identify a subject's personal aspects (Personal Aspects Profiling Process), using his or her eye responses. A subject's personal aspects include many things such as but not limited to: state of mind, level of stress, intensions, anxiety, fear, attentiveness, dislike, alertness, honesty, talents, concentration level, personal characteristics, emotions, preferences, mentality, drunkenness level, toxic level, background/memories or any combination thereof.

Eye responses are a complex response, which includes many different types of responses such as, but not limited to: fixation, gaze, saccades, convergence, rolling, pursuit, nystagmus, drift and micro-saccades, physiological nystagmus, blinking, pupil size, or any combination thereof. The eye movement response may include static characteristics dynamic characteristics or any combination thereof.

To understand how different eye movements can be used to characterize someone, a short review of the eye anatomy, physiology and functionality is given hereinafter. The retina of a human eye is not homogeneous. To allow for diurnal vision, the eye is divided into a large outer ring of highly light-sensitive but color-insensitive rods, and a comparatively small central region of lower light-sensitivity but color-sensitive cones, called the fovea. The outer ring provides peripheral vision, whereas all detailed observations of the surrounding world is made with the fovea, which must thus constantly be subjected to different parts of the viewed scene by successive fixations., Yarbus showed at 1967 (in “Eye movements during perception of complex objects, in L. A. Riggs, ed., and in “Eye Movements and Vision”, Plenum Press, New York, chapter VII, pp. 171-196) that the perception of a complex scene involves a complicated pattern of fixations, where the eye is held (fairly) still, and saccades, where the eye moves to foveate a new part of the scene. Saccades are the principal method for moving the eyes to a different part of the visual scene, and are sudden, rapid movements of the eyes. It takes about 100 ms to 300 ms to initiate a saccade, that is, from the time a stimulus is presented to the eye until the eye starts moving, and another 30 ms to 120 ms to complete the saccade. Usually, we are not conscious of this pattern; when perceiving a scene, the generation of this eye-gaze pattern is felt as an integral part of the perceiving process.

Fixation and saccades are not the only eye movement identified. Research literature, for example, “Eye tracking in advanced interface design, in W. Barfield & T. Furness, eds, ‘Advanced Interface Design and Virtual Environments’, Oxford University Press, Oxford, pp. 258-288”, by Jacob 1995, and “Visual Perception: physiology, psychology and ecology, 2nd edn, Lawrence Erlbaum Associates Ltd., Hove, UK”, by Bruce & Green 1990, identified six other different types of eye movements: (1) Convergence, a motion of both eyes relative to each other. This movement is normally the result of a moving stimulus: (2) Rolling is a rotational motion around an axis passing through the fovea-pupil axis. It is involuntary, and is influenced, among other things, by the angle of the neck; (3) Pursuit, a motion, which is a much smoother and slower than the saccade; it acts to keep a moving object foveated. It cannot be induced voluntarily, but requires a moving object in the visual field; (4) Nystagmus, is a pattern of eye movements that occur in response to the turning of the head (acceleration detected by the inner ear), or the viewing of a moving, repetitive pattern (the train window phenomenon). It consists of smooth ‘pursuit’ motion in one direction to follow a position in the scene, followed by a fast motion in the opposite direction to select a new position: (5) Drift and microsaccades, which are involuntary movements that occur during fixations, consist of slow drifts followed by very small saccades (microsaccades) that apparently have a drift-correcting function; and (6) Physiological nystagmus is a high-frequency oscillation of the eye (tremor) that serves to continuously shift the image on the retina, thus calling fresh retinal receptors into operation. Physiological nystagmus actually occurs during a fixation period, is involuntary and generally moves the eye less than 1°. Pupil size is another parameter, which is sometimes referred to as part of eye movement, since it is part of the vision process.

In addition to the six basic eye movements described above, more complex patterns involving eye movement have been recognized. These higher level and complex eye-movements display a clear connection between eye-movements and a person's personality and cognitive state.

Many research studies concluded that humans are generally interested in what they are looking at; that is, at least when they do spontaneous or task-relevant looking Exemplary publications include are “Perception and Information, Methuen, London, chapter 4: Information Acquisition, pp. 54-66” by Barber, P. J. & Legge, D. 1976; “An evaluation of an eye tracker as a device for computer input, in J. M. Carroll & P. P. Tanner, eds, ‘CHI+GI 1987 Conference Proceedings’, SIGCHI Bulletin, ACM, pp. 183-188. Special Issue”, by Ware & Mikaelian 1987); “The Human Interface: Where People and Computers Meet, Lifetime Learning Publications, Belmont, Calif. 94002”, by, Bolt 1984; and “The gaze selects informative details within pictures, Perception and Psychophysics 2, 547-552”, by Mackworth & Morandi 1967. Generally, the eyes are not attracted by the physical qualities of the items in the scene, but rather by how important the viewer would rate them. Thus during spontaneous or task-relevant looking, the direction of gaze is a good indication of what the observer is interested in (Barber & Legge (1976)). Similarly, the work done by Lang in 1993 indicates that, on average, the viewing time linearly correlates to the degree of the interest or attention an image elicits from an observer.

Furthermore, eye movements can also reflect the person's thought processes. Thus an observer's thoughts may be followed, to some extent, from records of his eye movements. For example it can easy be determined, from eye movement records, which elements attracted the observer's eye (and, consequently, his thought), in what order, and how often (Yarbus 1967, p. 190). Another example is a subject's “scan-path”. A scan-path is a pattern representing the course a subject's eyes take, when a scene is observed. The scan-path itself is a repeated in successive cycles. The subject's eyes stop and attend the most important parts of the scene, in his eyes, and skip the remaining part of the scene, creating a typical path. The image composition and the individual observer determine the scan-path, thus scan-paths are idiosyncratic (Barber & Legge 1976, p. 62).

In some embodiments the Profiling Process is done with full cooperation of the subject, in other situations, the identification process may be held without the knowledge of the subject.

In some embodiments the Profiling Process is combined with an identification process. Combining the Profiling personal aspects process with the ID-U identification process (US Patent: 20080104415) has the significant advantage of extracting both a user's identity and profile from the same signal at the same time, thus saving time and money. To our knowledge no other technology can provide such comprehensive information on a subject.

Scenarios, which may require extracting personal aspects of a subject, are numerous. One example is screening travelers in airports or other boarder stations for terrorists, smugglers, illegal passengers, etc. Another example is identifying and profiling employees at an airport. In this case the employees may include pilots, porters, service providers, stewardess, security officers etc. Another example may be as part of law enforcement activity such as investigation and interrogations. A different scenario could be for screening/interviewing employees to certain jobs or companies. In a similar manner the technology can be used to screen and allocate people in specific positions that best fit their talents and characteristics (in the army for example). Another example could be helping a subject “know himself better”, identify his skills and talents, and help himself choose his path wisely. A different application may be used in the electronic gaming industry. A player's profile may be prepared and used for the players benefit, or for his opponent to see. For example by calculating and displaying a player's stress level to his opponents, the game becomes more interesting and challenging.

The “Personal Aspects Profiling Process” as disclosed herein is based on the rich and diverse information embedded in a subject's eye-movement responses. From a subject's eye movement responses, many features can be extracted. Some of these features are robust to a subject's personal aspects, and therefore they may be used for identification tasks (US Patent: 20080104415). Other features are not robust; thus they reflect a subject's personal aspects. These features change when a subject's personal aspects change. For example, pupil activity changes when a person is under stress or intoxicated. Accordingly, by monitoring changes in pupil activity, one can detect stress. In a more general manner, by analyzing eye movement response, one can detect and profile a subject's personal aspects. Changes in a subject's personal aspects, may be evoked intentionally by specially designed stimuli, which are presented to the subject, alternatively they may be induced by outside uncontrolled factors (for example stress at work).

A subject's eye-movement activity may be acquired in any available method (ERG, Ober system, coil, video). In a preferred embodiment the eye-movements are acquired using a video camera.

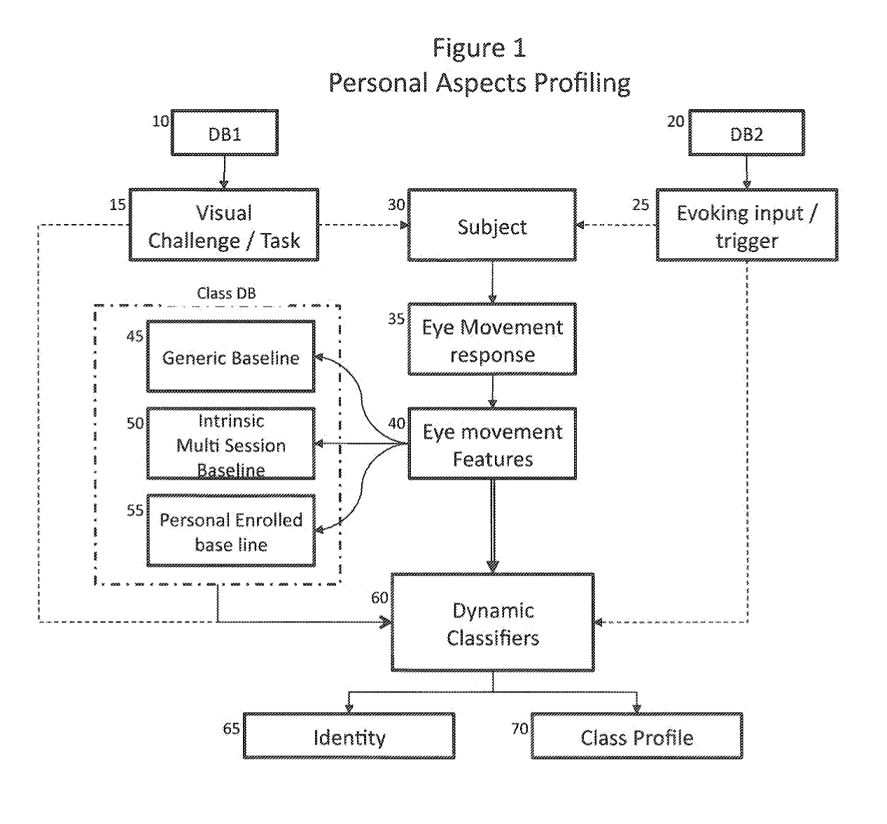

FIG. 1 discloses a block diagram of some preferred embodiments for implementing the Personal Aspects Profiling Process using eye responses. A subject (30) is subjected to an evoking input—evoking stimulus (25) or to a Visual challenge—visual stimulus (15) or to both of them. Both stimuli (15, 25) are selected according to the specific application required from corresponding databases (10, 20). Identifying if someone is stressed, or his mentality/background will usually require a different set of evoking stimuli. Examples for evoking inputs/stimuli (25): images, video, sound, smell, text, voice, music, touch, colors. However any other type of input, which influences the subject, is possible. The Visual challenge (15), can be any type of visual image that a user can see and visually respond to. For example a moving target, a fixed target, a static image/images, a moving image/images, a picture with multi items etc. This visual stimulus (15) is neutral, meaning it does not evoke any physiological or emotional reaction from the subject except for his eyes response, while he is watching or tracking it. The visual stimulus should initiate an eye movement response which includes both voluntary and automatic components. Furthermore, the visual challenge (15) should initiate eye responses, which are sensitive (influenced) to the subject's changes in his personal aspects.

In some embodiments, the two stimuli (15 and 25) can be the same. Thus the evoking stimulus is a visual stimulus which also get's the user's eyes to respond, creating eye movement responses. In other embodiments, there is no evoking stimulus at all, and only a visual stimulus is used. In these applications, it is assumed that subject is already in some kind of state, for example under stress, drunk or tired, thus no evoking input is required.

The subject's eye movements responses are acquired by any type of acquisition method, and from the eye response signal (35) a set of features are extracted (40). The extracted features (40) are entered to a class database. The features (40), the stimuli (15 and/or 25), and data from the class database (45, 50, 55) are used by a dynamic classifier (60), which uses the information to produce someone's Class Profiling (70), and in some embodiments his identification (65). The entire identification and profiling is done using one system and one method, which is based on eye responses.

Furthermore, in accordance with some preferred embodiments of the present invention profiling a subject's personal aspects includes analysis of his eye response to a series of different evoking stimuli (15) during a single session, thus creating an intrinsic multi session base line. The extracted features (40) will be analyzed using the intrinsic multi session baseline (50) and the dynamic classifier (60).

Furthermore, in accordance with some preferred embodiments of the present invention profiling a subject's personal aspects includes analysis of his eye movement response to a set of evoked stimuli (15). The extracted features (40) will be analyzed using a genric baseline (45), which was calculated previously, and which reflects typical values of the different features correlated to different personal aspects. This information together with the stimuli and responses will be used by the classifier (60) to determine the subject's identity (65) and class profile (70).

Furthermore, in accordance with some preferred embodiments of the present invention profiling a subject's personal aspects includes analysis of his eye movement response to a set of evoked stimuli (15). The extracted features (40) will be analyzed using the subject's personal enrolment baselines (55), which were calculated previously in an enrollment stage, and which reflects typical values of his personal identity and personal aspects. This information together with the stimuli and responses will be used by the classifier (60) to determine the subject's identity (65) and class profile (70).

The exact methodology and embodiment used, depends partially on the exact application and identification required.

In some applications, evoking stimuli, which may create a specific response, will be given to the subject each time he approaches the system. Thus changes in his response to a particular visual stimulus will indicate changes in the subject's personal aspects.

In other applications a set of different evoking stimulus will be given to the subject, and his eye-movement responses to the visual stimuli will be analyzed and compared. This comparison will enable detecting a subject's personal aspects.

In yet other applications a set of different evoking stimulus will be given to the subject, and his eye-movement responses for the different stimuli will compared to typical responses from a database. Other applications will use some eye-movement responses as a subject's base line and compare them to other eye-movement response when tested.

To better understand the process several specific example embodiments are presented.

In the following examples stress of a subject is detected by analyzing his eye-movement response to a visual stimulus. The stressed conditions can be evoked by the system using any kind of evoking stimuli. Alternatively the stress conditions could be caused by everyday events, which the system does not recognize and control. Features are extracted from the subject's eye-response. The same methodology can used for other personal aspects in a similar manner.

In one preferred embodiment the same visual stimuli are given to the subject under “relaxed conditions” (Baseline conditions) and under “potentially stressed conditions” (PSC) during a single session. This requires using evoking stimuli in addition to the visual stimuli (the evoking stimuli and the visual stimuli can be the same). By comparing the user's features at Baseline conditions to those at PSC, one can detect, which evoking stimuli cause the subject stress. In this embodiment a users is profiled using his intrinsic multi session base-line. He does not need previous enrollment to the system.

A somewhat different approach may include enrolling the subject to the system, exposing him to baseline and stress conditions, acquiring his eye-response, extracting features, and saving the subjects Baseline and PSC stress features in a database (personal enrolled baseline). Thus now, by analyzing and comparing the subject's current eye response and features to his baseline and stress values, stress of the subject can be detected.

In another embodiment a generic baseline values and stress values are predefined for a set of selected features. When testing, if a subject is under stress, his sampled eye-movement features are compared to the predefined baseline values, and thus it can be determined if he is under stress.

In another preferred embodiment, a subject's personal aspects (stress, recognition, lying, familiarity, dislike, contempt etc.), are detected, using his eye-movement response to a set of evoking stimuli images. To better understand the methodology, an example using eye-movement features based on pupil dilation dynamics (PDD) is used. However the same method can be applied to other eye-movement features as well examples including but limited to: quality of tracking, delay in tracking, overshoot, undershoot, blinking, fixation quality etc.

The pupil of any person continuously changes its diameter. These changes are due to changes in illumination, but they also reflect different attributes of the subject's current state (mental, cognitive, concentration, stress, familiarity, laying etc.). In order to detect a subject's reaction/state to an evoking stimulus, it is necessary to differentiate between a “normal” ongoing PDD activity and an intentionally evoked PDD, which was caused by his emotional reactions to an evoking stimulus or by uncontrolled changing conditions. This is done by analyzing the PDD signal.

The following methodology is a suggested method for analyzing the PDD signal, but other methods may be used to achieve the similar results.

The first step is aimed at establishing a baseline PDD. The baseline PDD can be personal or generic in nature. For establishing a generic/personal PDD baseline, a group of subjects/a subject is presented with “standard stimuli”, for example unfamiliar and non disturbing neutral images. A video camera acquires the subject's eye-response to the stimuli images. These signals will be used to define the baseline PDD signal. Analysis of the baseline PDD will enable characterizing such signal. For example, blinking activity creates a PDD signal. Blinking is characterized by a signal with specific dilation/expansion velocity, acceleration, duration and shape. Thus blinking zones can be detected anywhere within the PDD signal, and referred to as part of a baseline PDD. This activity is not correlated to the stimulus. The same process is repeated with other ongoing baseline activities such as PDD segments correlated to reading activity, illumination changes, activities which require considerable concentration, etc. Some of these PDD responses are correlated to a stimulus other are not. Using these baseline PDD segments, a generic/personal baseline PDD can be characterized.

The next step includes superimposing evoked PDD signals onto the baseline PDD. One may create evoked PDD activity in many ways. For example, by showing a subject a set of images, which may be disturbing or familiar to him. Another example is asking the subject questions which we know may be disturbing or even forcing the subject to lie.

The evoked PDD segments represent situations where the subject may have responded to the stimuli. Since we are dealing with evoked stimuli the potentially evoked PDD segments must be synchronized in time with the exposure to the stimuli. Thus only segments in specific time slots are potential for being evoked PDD segments. Only these potential segments are analyzed at this stage. Using these segments the different evoked response are mapped and characterized.

When one wants to test a subject, he is exposed to stimuli images, and his PDD signal is analyzed. Using the baseline PDD, it is now possible to identify if the subject reacted to specific stimuli in patterns, which are characteristic to stress, lying, dislike, distress, etc.

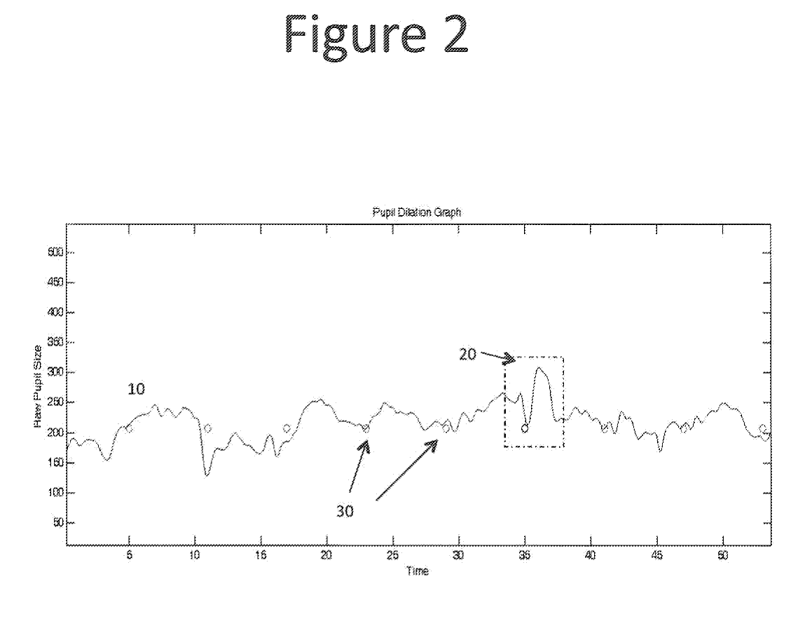

The following experiment is an example of characterizing and mapping a PDD signal correlated to recognition and stress. In this example, a subject is shown 9 images of cards on a screen, and is asked to choose one card. The operator then displays the cards one by one, and asks the subject if the present card is the one selected. The subject is asked to say no each time, he is asked. This means that subject is forced to lie once. FIG. 2 shows a graph of the PDD (10) of such an experiment. The 9 small circles (30) represent the instance where the card appeared on the screen and the subject was forced to answer the question. A window (20) superimposed on the PDD signal (10) represents the instance where the selected card was presented, and the subject was forced to lie. It can be seen that when the subject was forced to lie, his PDD signal (10) shows a distinct and correlated signal different from the baseline PDD activity. The pupil response to lying is characterized by several parameters such as a specific delay, a typical duration of the dilation and contraction, and a typical morphological shape of the peak. These can all bee seen in window 20.

Once the PDD signal following the onset of a lie is characterized, and the baseline PDD is mapped, one can use the PDD to detect stress and lies.

In a preferred embodiment, eye movement features were selected, and baseline classes were obtained by comparing eye movement responses and features to readings made by a Galvanic skin response device (which is the standard signal of the polygraph), while subjecting a subject to an evoking stimuli.

Galvanic skin response (GSR) is a method of measuring the electrical resistance of the skin. There is a relationship between sympathetic activity and emotional arousal, although one cannot identify the specific emotion being elicited; Fear, anger & startle response are all among the emotions which may produce similar GSR responses. The change is caused by the degree to which a person's sweat glands are active: Psychological stress tends to make the glands more active and this lowers the skin's resistance.

In one embodiment a presentation including both audio and visual stimuli was presented to a subject. The stimuli were designed to evoke an emotional response from the subject. The subject's eye movements were acquired using a camera, and a set of features extracted. The subject's GSR signal was recorded at the same time. In yet another embodiment, non-visual evoking stimuli were presented to a subject, while he was watching a visual target moving in a predefined pattern. The subject's eye movement response to the moving target was acquired using a camera, and a set of features extracted. The subject's GSR signal was recorded at the same time.

In yet another embodiment, when the subject is subjected to an evoking stimulus of any kind, eye movement patterns and behaviors, which are typical of stress, are detected within the eye-movement signal.

A set of features, which were correlated with GSR signal were derived from the eye-movement signal. Examples of such features are pupil dilation and contraction behavior, changes in saccadic movements, changes in frequency content of the signal, quality of tracking the target; overshoot/undershoot behavior, and quality and quantity of fixations.

While specific embodiments were described, this was done as means for helping to clarify, how the invention works. The detailed embodiments are merely examples of the disclosed system and method. This does not imply any limitation on the scope of the disclosed invention. Applicant acknowledges that many other embodiments are possible.

What we claim is:

1. A method for profiling a personal aspect of a subject, the method comprising the steps of:
subjecting a subject to:
at least one visual stimulus selected from a stimulus database comprising a multiplicity of stimuli; and to
at least one evoking stimulus selected from a database of evoking stimuli;
acquiring at least one eye response from said subject to said visual stimulus; and
processing said eye response, said visual stimulus and said evoking stimulus for profiling at least one personal aspect of said subject.

2. The method of claim 1, wherein said evoking stimulus and said visual stimulus are the same stimulus.

3. The method of claim 1, wherein features are extracted from said eye response.

4. The method of claim 1, wherein said processing comprises using a class database.

5. The method of claim 4, wherein said class database comprises either a generic baseline, or an intrinsic baseline or a personal enrolled baseline or any combination thereof.

6. The method of claim 4, wherein said processing said eye response and said visual stimulus comprises pattern recognition analysis.

7. The method of claim 1, wherein said visual stimulus comprises a target moving and halting in a predefined trajectory.

8. The method of claim 1, wherein said eye response comprise fixation, gaze, saccades, convergence, rolling, pursuit, nystagmus, drift and microsaccades, physiological nystagmus pupil size, pupil dynamic, blinking or any combination thereof.

9. The method of claim 1, wherein said personal aspect comprises state of mind, level of stress, intensions, anxiety, fear, attentiveness, dislike, alertness, honesty, talents, concentration level, personal characteristics, emotions, preferences, mentality, drunkenness level, toxic level, background/memories or any combination thereof.

10. The method of claim 1, wherein said processing said eye response and said visual stimulus are used for identifying a user and profiling at least one personal aspect of said subject.

11. A system for profiling a personal aspect of a subject, the system comprising:
a processor adapted to select:
at least one visual stimulus from a database comprising a multiplicity of visual stimuli; and
at least one evoking stimulus from a database comprising a multiplicity of evoking stimuli;
and
at least one sensor adapted to acquire at least one eye response of a subject to said visual stimulus,
wherein said processor is further adapted to perform processing and analysis of said visual stimulus, said evoking stimulus and said eye response, for profiling at least one personal aspect of said subject.

12. The system of claim 11 wherein said evoking stimulus and said visual stimulus are the same stimulus.

13. The system of claim 11, wherein said processor is further adapted to extract features from said eye response.

14. The system of claim 11, wherein said processor is adapted to use a class database.

15. The system of claim 14, wherein said class database comprises either a generic baseline, or an intrinsic baseline or a personal enrolled baseline or any combination thereof.

16. The system of claim 14, wherein said processor is further adapted to perform pattern recognition analysis.

17. The system of claim 11, wherein said visual stimulus comprises a target moving and halting in a predefined trajectory.

18. The system of claim 11, wherein said eye response comprises fixation, gaze, saccades, convergence, rolling, pursuit, nystagmus, drift and microsaccades, physiological nystagmus pupil size, pupil dynamic, blinking or any combination thereof.

19. The system of claim 11, wherein said personal aspect comprises state of mind, level of stress, intensions, anxiety, fear, attentiveness, dislike, alertness, honesty, talents, concentration level, personal characteristics, emotions, preferences, mentality, drunkenness level, toxic level, background/memories or any combination thereof.

20. The system of claim 11, wherein said processor is further adapted to establish the subject's identity and to profile at least one personal aspect of said subject.
